(19) United States
(12) Patent Application Publication
(10) Pub. No.: US 2018/0110803 A1
(43) Pub. Date: Apr. 26, 2018

(54) Neutral Electrolyzed Water and Uses Thereof

(71) Applicants: Juan Paz Garcia, Colima (MX); Brenda Astrid Paz Michel, Mexico D.F. (MX); Mario Valbunea Moctezuma, Mexico D.F. (MX); Ivan Delgado Enciso, Colima (MX)

(72) Inventors: Juan Paz Garcia, Colima (MX); Brenda Astrid Paz Michel, Mexico D.F. (MX); Mario Valbunea Moctezuma, Mexico D.F. (MX); Ivan Delgado Enciso, Colima (MX)

(21) Appl. No.: 15/791,927

(22) Filed: Oct. 24, 2017

(30) Foreign Application Priority Data
Oct. 24, 2016 (MX) MX/A/2016/013933

(51) Int. Cl. A61K 33/14; A61K 33/20; A61K 9/06; A61K 9/08

(52) U.S. Cl. CPC A61K 33/14; A61K 9/08; A61K 9/06; A61K 33/20

(57) ABSTRACT

Provided herein is a neutral electrolyzed water or activated saline or gel formulation thereof. The neutral electrolyzed water comprises active species of chlorine and oxygen [Cl/O] at a concentration of about 0.00001% (0.1 ppm) to about 0.004% (40 ppm) and sodium chloride at a concentration of about 3% or less in reverse osmosis water. The neutral electrolyzed water or formulations are useful when administered or delivered to a subject for reducing bleeding or inducing hemostasis during a procedure, for reducing inflammation, for reducing pain, for regenerating or repairing a tissue or organ, and/or for modulating activity of the immune system to control chronic inflammation and/or exacerbated oxidant stress and for inducing a therapeutic effect.

### CROSS-REFERENCE TO RELATED APPLICATIONS

This non-provisional application claims benefit of priority under 35 U.S.C. § 119(a) of Mexican application number MX/a/2016/013933, filed Oct. 24, 2016, the entirety of which is hereby incorporated by reference.

### BACKGROUND OF THE INVENTION

### Field of the Invention

The present invention generally relates to the field of uses of electrolyzed or activated saline waters. Particularly the present invention relates to the therapeutic use of neutral electrolyzed or activated saline water (ASW) as an immunomodulatory-homeostatic agent and/or regenerative agent.

### Description of the Related Art

Electrolyzed water has been used as a topical antiseptic or disinfectant or as a microbicide to treat microbial infections or antigen-related infections in warm blooded animals. For example U.S. Pat. No. 5,731,008 discloses a microbiocidal isotonic solution of an electrolyzed saline that contains regulated amounts of ozone of about 5-100 mg/L and active chlorine species of 5-300 ppm. The microbiocidal isotonic solution can be intravenously administered alone or with anti-oxidant enzymes or cofactors and a mitotic inhibitor for the treatment of microbial infections.

U.S. Pat. No. 5,334,383 discloses electrically hydrolyzed salines as microbicides for the treatment of antigen related infections in cardiomyopathy and multiple sclerosis. The electrically hydrolyzed isotonic saline, containing between 100-300 ppm of microbicidal agents like chloride ions along with other hydrolysis products balanced with an amount of hypertonic saline, was injected intravenously after I.V. administration of colchicine and before administration of one of superoxide dismutase, myeloperoxidase, glutathione peroxidase, glutathione, catalase, ascorbic acid and sodium ascorbate.

U.S. Pat. No. 5,674,537 discloses an electrolyzed saline solution containing concentrated amounts of ozone and chlorine species for in vivo and in vitro treatment of microbial infections. The ozone content is in the range of about 5-100 mg/L and the chlorine species content is in the range of about 5-300 ppm. The solution is used in in vitro treatments of infected whole blood, blood cells or plasma to reduce contamination and may be administered to warm blooded animals for same purposes.

Some other examples about electrolyzed saline solutions can be found in previous art describing their utility, only as microbicides for treatment of infectious diseases or antisepsis of contaminated tissues or unanimated surfaces.

Warm-blooded animals, particularly, a human, are increasingly subjected to exacerbated stress conditions, whether pathological or non-pathological (social, occupational or environmental), such that disorders or imbalances occur; i.e. homeostatic disruptions, that generates medical conditions, diseases, co-morbidities or aggravation of existing pathologies. Generally, the immediate response to such imbalances is an immune response that manifests as inflammatory profile, pain, compromised tissues and cell functions and/or a dysregulation of homeostasis resulting in non-infectious pathologies, sleeplessness, fatigue, depression, and/or obesity. Tissue inflammation and dysregulation of homeostasis also is intrinsically related to intense physical activity and exercise.

Many current treatments of conditions resulting from inflammation or involving an inflammatory component are not curative and require that dosages be increased over time which increases the danger of overdosing the patient. Most recent therapies include, for example, administration of monoclonal antibodies that selectively blocks certain pro-inflammatory enzymes (pro-inflammatory cytokines); such therapies are significantly expensive and also had demonstrated immunologic imbalance along with increased susceptibility to infections and tissue damage. Alternatively, certain physical or chemical stimuli had demonstrated to induce adaptive response to oxidative stress and so to inflammatory response in diverse mammal cells or murine models. This mechanism may offer an alternative to control and/or treat non-infectious inflammatory pathologies or conditions, since could induce immunologic system modulation. An electrolyzed or activated saline water (ASW) may work as such chemical stimulus providing adaptive protection against oxidative stress and so inflammatory profile, modulating immunologic response in a subject.

Electrolyzed waters are synthesized from controlled electrolysis of salines as sodium chloride. REDOX reactions occur in electrolytic cell, providing an electrolyzed water comprising different concentrations of active oxidant species. While active oxidant species work as effective disinfectants and antiseptics when present in high concentration, it is contemplated that active oxidant species in low concentrations may act as inducers of adaptive responses to oxidative stress.

Therefore, there is a need in the art for an electrolyzed water that is not a microbicide but an immunomodulatory-homeostatic agent and/or regenerative agent, effective to modulate an activity of the immune system to induce a beneficial or therapeutic effect against a pathological or non-pathological condition or stress-related condition. Particularly, the prior art is deficient in a neutral electrolyzed water comprising active species of chlorine and oxygen [Cl/O] or formulation thereof that modulates immune system activity in a subject due to, for example, to modulate inflammatory and/or oxidative profile, to reduce pain, to induce hemostasis, to regenerate compromised tissue, or to restore homeostasis. The present invention fulfills this long-standing need and desire in the art.

### SUMMARY OF THE INVENTION

The present invention is directed to a neutral electrolyzed water that induces modulation of immune system, haemostasis, tissue repair, analgesia and an anti-inflammatory effect. The neutral electrolyzed water comprises, in reverse osmosis water, active oxidant species of chlorine and oxygen [Cl/O] at a concentration of about 0.00001% or 0.1 ppm to about 0.004% or 40 ppm and sodium chloride at a concentration of about 3% or less, an oxidation-reduction potential of 500-980 mV, pH values of 6.5-7.5 and an osmolarity of 30-350 mOsm/L. The active oxidant species of chlorine and oxygen [Cl/O] comprise HClOₓ, wherein X is 1 to 4, ClOₓ⁻, wherein X is 0 to 4, ClO₂, O_{y}, wherein Y is 2 or 3, Cl₂, H₂, or H₂O₂, or combinations thereof.

The present invention also is directed to a formulation comprising the neutral electrolyzed water described herein in a saline vehicle or in a gel. An example is activated saline water or a neutral activated saline.

The present invention is directed further to a method for reducing bleeding in a subject during a procedure performed thereon. The method comprises the step of administering one or more times to the subject an amount of the neutral electrolyzed water described herein effective to induce hemostasis therein, thereby reducing bleeding during the procedure.

The present invention is directed further still to a method for reducing neuropathic pain or for alleviating palsy in a subject in need thereof. The method comprises the step of administering one or more times to the subject an amount of the neutral electrolyzed water described herein effective to reduce the neuropathic pain or to alleviate the palsy in the subject.

The present invention is directed further still to a method for modulating an activity of the immune system to induce a therapeutic effect in a subject in need thereof. The method comprises the step of administering one or more times to the subject an amount of the neutral electrolyzed water described herein such that the activity of the immune system is regulated effectively to modulate inflammatory and oxidant profiles, to improve response against stress-related conditions, to stimulate repair of a tissue or an injured organ, to reduce pain, or a combination thereof in the subject.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1F show MDCK cells exposed to ASW with different concentrations of [Cl/O]. In FIG. 1A [Cl/O] is 0 (control), in FIG. 1B [Cl/O] is 50 ppm, in FIG. 1C [Cl/O] is 30 ppm, in FIG. 1D [Cl/O] is 15 ppm, in FIG. 1E [Cl/O] is 7.5 ppm, and in FIG. 1F [Cl/O] is 4 ppm.

FIG. 2 is a cell viability curve of different concentrations of ASW over human fibroblasts.

FIG. 3 illustrates bleeding evolution after treatment of injury with distilled water (DW), normal saline (NS) and ASW.

FIG. 4 illustrates the nociceptive response of Wistar rats treated with normal saline (NS), ASW, 10 ppm, ASW, 20 ppm or ketorolac (KET), during a formalin model of inflammatory pain.

FIGS. 5A-5B are biopsies of auricle graft 14 days post-initial treatment with normal saline showing cicatrization process. FIG. 5A shows dense collagen fiber and FIG. 5B shows dense collagen fibers and tissue discontinuity.

FIGS. 6A-6B are biopsies of auricle graft 14 days post-initial treatment with ASW gel showing tissue regeneration process. FIG. 6A shows tissue regeneration with angiogenesis process and FIG. 6B shows tissue regeneration including hair follicle (arrow).

FIGS. 7A-7B shows a female patient irrigated with ASW instead of NS during maxillary advancement. The tissue quality and limited inflammation process are noticeable immediately after surgery (FIG. 7A) and 48 hours post-surgery (FIG. 7B).

FIG. 8 is an x-ray image from a patient with maxillary advancement at 4 weeks post-surgery. Notice the formation of bone callus (arrow).

FIGS. 9A-9C illustrate a retroauricular infection of a female patient before treatment with ASW (FIG. 9A), with the wound cleaned up with ASW and filled with ASW gel (FIG. 9B) and showing the retroauricular lesion, 20 days post-ASW treatment (FIG. 9C). The absence of significant inflammatory process in FIG. 9B and the fast wound healing in FIG. 9B are apparent.

FIGS. 10A-10D illustrate an Infected apical cyst in the left nasolabial region of female patient (FIG. 10A), the wound cleaned up with ASW and filled with ASW gel (FIG. 10B), wound evolution at 9 days post-surgical management (FIG. 10C), and wound closure at 21 days post-treatment (FIG. 10D). The absence of significant inflammatory process in FIG. 10B and the wound advance and the presence of fibroblastoid-like tissue all over the wound border in FIG. 10C are apparent.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “activated saline water” or “ASW” refers to a formulation of the neutral electrolyzed water in a vehicle such as saline or a gel formulation of the neutral electrolyzed water.

As used herein, the term “compromised tissue” or “injured tissue” refers to a loss of tissue integrity or formation of a lesion via the effects of a pathophysiological condition, for example, such as caused by rheumatoid arthritis, a cancer, a tumor, a cyst, or inflammation or caused by an injury or wound.

As used herein, the term “systemic” refers generally to the whole body of a subject.

As used herein, the term “subject” refers generally to any recipient of the neutral electrolyzed water or activated saline water described herein.

In one embodiment of the present invention, there is provided a neutral electrolyzed water that induces modulation of immune system, haemostasis, tissue repair, analgesia and an anti-inflamatory effect, comprising, in reverse osmosis water, an active oxidant species of chlorine and oxygen [Cl/O] comprising HClOₓ, wherein X is 1 to 4, ClOₓ⁻, wherein X is 0 to 4, ClO₂, O_{y}, wherein Y is 2 or 3, Cl₂, H₂, or H₂O₂, or combinations thereof at a concentration of about 0.00001% or 0.1 ppm to about 0.004% or 40 ppm; and sodium chloride at a concentration of about 3% or less.

In this embodiment the pH may be about 6.5 to about 7.5. Also, the oxidation-reduction potential may be about 500 mV to about 980 mV. In addition the osmolarity may be about 30 mOsm/L to about 50 mOsm/L.

In one aspect of this embodiment the [Cl/O] may be about 0.001% to about 0.004%, the oxidation-reduction potential may be about +750 to about +980 mV and the osmolarity may be about 30 mOsm/L to about 100 mOsm/L. In another aspect of this embodiment the [Cl/O] may be about 0.0005% to about 0.002%, the oxidation-reduction potential may be about +600 to about +900 mV and the osmolarity may be about 30 mOsm/L to about 150 mOsm/L. In yet another aspect the [Cl/O] may be about 0.00001% to about 0.001%, the oxidation-reduction potential may be about +500 to about +650 mV and the osmolarity may be about 150 mOsm/L to about 350 mOsm/L.

In a related embodiment there is provided a formulation comprising the neutral electrolyzed water in a saline vehicle or a gel.

In another embodiment of the present invention there is provided a method for reducing bleeding in a subject during a procedure performed thereon, comprising the step of administering one or more times to the subject an amount of the neutral electrolyzed water, as described supra, effective to induce hemostasis therein, thereby reducing bleeding during the procedure.

In this embodiment the neutral electrolyzed water may have a pH of about 6.5 to about 7.5, a [Cl/O] of about 0.001% to about 0.004%, an oxidation-reduction potential of about +750 to about +980 mV and an osmolarity of about 30 mOsm/L to about 100 mOsm/L. Also, in this embodiment the step of administering the neutral electrolyzed water may induce repair of an injured tissue, reduces inflammation or reduces pain or a combination thereof. In addition the step of administering the neutral electrolyzed water induces repair of an injured tissue, reduces inflammation or reduces pain or a combination thereof. Particularly, the injured tissue that is repaired is skin, connective tissue, muscle, or periodontium. Furthermore the procedure performed on the subject may be a surgical procedure, a dental procedure or a medical procedure to treat a lesion, a wound, a burn or a site of infection.

In yet another embodiment of the present invention there is provided a method for reducing neuropathic pain or for alleviating palsy in a subject in need thereof, comprising the step of administering one or more times to the subject an amount of the neutral electrolyzed water, as described supra, effective to reduce the neuropathic pain or to alleviate the palsy in the subject.

In this embodiment the neutral electrolyzed water may have a pH of about 6.5 to about 7.5, a [Cl/O] of about 0.0005% to about 0.002%, an oxidation-reduction potential of about +600 to about +900 mV and an osmolarity of about 30 mOsm/L to about 150 mOsm/L. Also in this embodiment the step of administering the neutral electrolyzed water may induce regeneration of a tissue, reduces inflammation or a combination thereof. Particularly, the regenerated tissue may be nerve fibers, myelin or myelin axons. In addition, the subject may have a neuralgia, diabetic neuropathy, Bell's palsy, or a viral disease.

In yet another embodiment of the present invention there is provided a method for modulating an activity of the immune system to induce a therapeutic effect in a subject in need thereof, comprising the step of administering one or more times to the subject an amount of the neutral electrolyzed water of claim 1 to the subject such that the activity of the immune system is regulated effectively to modulate inflammatory and oxidant profiles, to improve response against stress-related conditions, to stimulate repair of a tissue or an injured organ, to reduce pain or a combination thereof in the subject.

In this embodiment the neutral electrolyzed water may have a pH of about 6.5 to about 7.5, a [Cl/O] of about 0.00001% to about 0.001%, an oxidation-reduction potential of about +500 to about +650 mV and an osmolarity of about 150 mOsm/L to about 350 mOsm/L. Also in this embodiment the injured tissue or organ that is repaired is skin, oral tissues, bone marrow, myelin, sexual gonads, kidney, liver, pancreas, muscle, or a combination thereof. In addition the subject may have chronic chikungunya, rheumatoid arthritis, arthralgia, cirrhosis, hepatitis, diabetes, migraine, fatigue, stress, metabolic syndrome, polycystic ovary, azoospermia, renal insufficiency, esophageal varices, kidney damage, liver damage, medullary hypoplasia, neutropenic fever, lupus, depression, irritable bowel syndrome, obesity, extreme fatigue from physical activity, sleep disorders, asthma, allergies, infertility or a combination thereof.

Provided herein is a neutral electrolyzed water or formulations thereof and methods of its use as an immunomodulatory-homeostatic agent and/or regenerative agent. The formulations of the neutral electrolyzed water may comprise the neutral electrolyzed water in a vehicle, such as, but not limited to, a saline or a gel. These saline formulations are preferable for intravenous, intramuscular, or intraneural or other parenteral routes of administration. Alternatively, the neutral electrolyzed water and activated saline water formulation may be delivered by directly irrigating or contacting an area of interest in the subject. A gel formulation of the neutral electrolyzed water may be applied directly to cover or fill the area of interest in the subject. Formulating a substance in a saline or as a gel is well-known in the art.

The neutral electrolyzed water or formulations thereof are useful in methods to modulate an activity of the immune system to induce a therapeutic or beneficial effect in a subject. For example, modulation of the activity has a therapeutic effect by reducing bleeding or inducing hemostasis during a procedure in a subject. Examples of the procedure for which the neutral electrolyzed water or formulations are beneficial are, but not limited to, a surgical procedure, a dental procedure or a medical procedure to treat a lesion, a wound, a burn, or a site of infection.

Also, other beneficial effects are reducing inflammation and/or nociceptive or neuropathic pain, improving an inflammatory profile and/or response against stress-related conditions, stimulating repair and regeneration of compromised tissue. Modulating an activity of the immune system also is effective to restore homeostasis. These therapeutic or beneficial or improved effects may be produced in a subject with a condition including, but not limited to, rheumatoid arthritis, cirrhosis, hepatitis, diabetes, an infection, a lesion, a wound, a virus, arthralgia, a neuralgia, Bell's palsy, infertility, fatigue, muscle injury from physical activity, or a combination thereof.

One of ordinary skill in the art is well able to determine dose or dosage and a dosing regimen depending on the procedure to be performed or the condition for which modulation of an activity of the immune system is desired. The neutral electrolyzed water or formulations described herein may be administered or delivered singly or in concurrent or sequential combination with one or more drugs. It is demonstrated herein that usage of the neutral electrolyzed water or formulations can reduce the need for or reduce the dosage of previously taken drugs.

Representative examples of activated saline water and the physicochemical characteristics and exemplary uses are shown in Table 1.

TABLE 1

| Physicochemical Characteristics of ASW | | | | | |
|---|---|---|---|---|---|
| | ORP | [Cl/O] | Osmolarity | Administration | |
| pH | (mV) | (%) | (mOsm/L) | route | Uses |
| 6.5 | +750 | 0.001 | 30 | Irrigation of | To increase tissue |
| to | to | to | to | tissue, wounds | regeneration and to |
| 7.5 | +980 | 0.004 | 100 | and exposed | induce hemostatic, anti- |
| | | | | organs | inflammatory and |
| | | | | | analgesic effects, for |
| | | | | | example in wound care, |
| | | | | | treatment of all type of |
| | | | | | burns and during minor |
| | | | | | and major surgeries |
| | +600 | 0.0005 | 30 | Intra-muscular | To reduce inflammation |
| | to | to | to | and mainly | and pain and to induce |
| | +900 | 0.002 | 150 | intra-neural | regeneration of myelin in |
| | | | | | trigeminal neuralgia and |
| | | | | | facial palsy (Bell's palsy) |
| | +500 | 0.00001 | 150 | Intravenous | To modulate immune |
| | to | to | to | | system and, thereby, to |
| | +650 | 0.001 | 350 | | modulate inflammatory |
| | | | | | profile, to increase |
| | | | | | response against |
| | | | | | infections and to induce |
| | | | | | cell, tissue and organ |
| | | | | | repair. Examples of |
| | | | | | diseases are those |
| | | | | | derived from |
| | | | | | inflammatory profile |
| | | | | | and/or immune system |
| | | | | | compromise such as |
| | | | | | chronic chikungunya, |
| | | | | | rheumatoid arthritis, C |
| | | | | | hepatitis, hepatic |
| | | | | | cirrhosis, arthralgia, |
| | | | | | steatohepatitis, type II |
| | | | | | diabetes, migraine, |
| | | | | | lupus, metabolic |
| | | | | | syndrome, polycystic |
| | | | | | ovary, azoospermia, |
| | | | | | renal insufficiency, |
| | | | | | esophageal varices, |
| | | | | | kidney damage, liver |
| | | | | | damage, chemotherapy |
| | | | | | or radiotherapy |
| | | | | | overdose, medullary |
| | | | | | hypoplasia, neutropenic |
| | | | | | fever, depression, |
| | | | | | detoxification therapy |
| | | | | | (illegal drugs abuse), |
| | | | | | irritable bowel |
| | | | | | syndrome, diabetic |
| | | | | | neuropathy, obesity, |
| | | | | | exacerbated or chronic |
| | | | | | fatigue and stress, |
| | | | | | asthma, allergies, sleep |
| | | | | | disorders and diseases |
| | | | | | related with an infection. |

The following example(s) are given for the purpose of illustrating various embodiments of the invention and are not meant to limit the present invention in any fashion.

### Example 1

### General Synthesis for Activated Saline Water

The electrolytic chamber is described in Mexican patent 330845. Initially a sodium chloride solution, consisting of 600-2000 ppm of NaCl, is prepared by mixing highly purified water with a 15% solution of sodium chloride. Such solution is conducted into the electrolytic chamber, operating at 400 Amps and 16-20 Volts, with a 9-15 L/min flux. Electrochemical properties (pH, ORP and active species of chlorine and oxygen) of the final electrolyzed mixture are adjusted to desired values and the pre-sterilization process is achieved by filtration. Then the activated saline water is packed in glass ampules and the sterilization process is completed by dry heat and pressure.

Specific physicochemical characteristics, at the moment of chemical analysis, for a specific type of an activated saline water are: pH=6.8, ORP=887 mV, NaCl=1820 ppm, [Cl/O]=40 ppm (Constituted by: HOCl/OCl⁻=31 ppm; Cl_{2(aq)}=3.1 ppm; ClO₂⁻/ClO₃⁻=1.2 ppm; O₂/O₃=2.6 ppm; ClO₄⁻/H₂O₂/ClO₂/other active species of chlorine and oxygen=2.1).

### Example 2

### In Vitro Evaluation of Activated Saline Water Cytotoxicity and Proliferative Effect Evidence

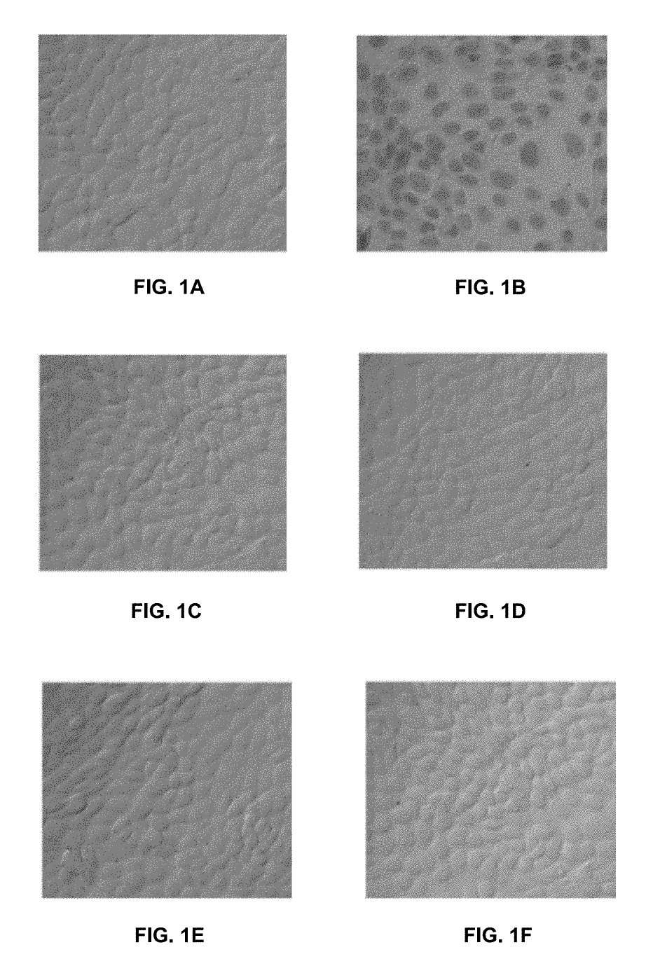

Activated saline water with different concentrations of active species of chlorine and oxygen [Cl/O] was evaluated over different cell types to determine potential cytotoxicity. Activated saline waters with pH values of 6.8-7.3, ORP values between 700-900 mV and [Cl/O] of 50, 30, 15, 7.5 and 4 ppm were evaluated over three different ATCC lines, Madin-Darby Canine Kidney (MDCK), intestinal epithelial cells, (IEC) and CaCo-2, during 30 minutes at room temperature. Activated saline water was mixed with non-supplemented DMEM media to achieve the described [Cl/O] concentrations and confluent cell lines were exposed to each mixture. Cells were grown in culture plates previously packed with coverslips and incubated at 37 degrees C. in 5% CO₂ atmosphere during 30 minutes with each activated saline water. Cells were then washed off two times with non-supplemented DMEM and stained with trypan blue (0.4% in PBS) for 1 minute and washed again 3 times with the same culture medium. Finally cells were fixed with formalin (10% in PBS) and mounted on slides with VECTASHIELD. Slides were analyzed by phase contrast microscopy. Identical results were obtained with all cellular lines. As an example, FIGS. 1A-1F show MDCK cells exposed to, respectively, non-supplemented DMEM (control), activated saline water with [Cl/O] of 50 ppm, activated saline water with [Cl/O] of 30 ppm, activated saline water with [Cl/O] of 15 ppm, activated saline water with [Cl/O] of 7.5 ppm and activated saline water with [Cl/O] of 4 ppm. Clearly the only affected cells were those treated with [Cl/O]=50 ppm, proving that is safe to apply activated saline water over exposed tissues, at concentrations below 30 ppm.

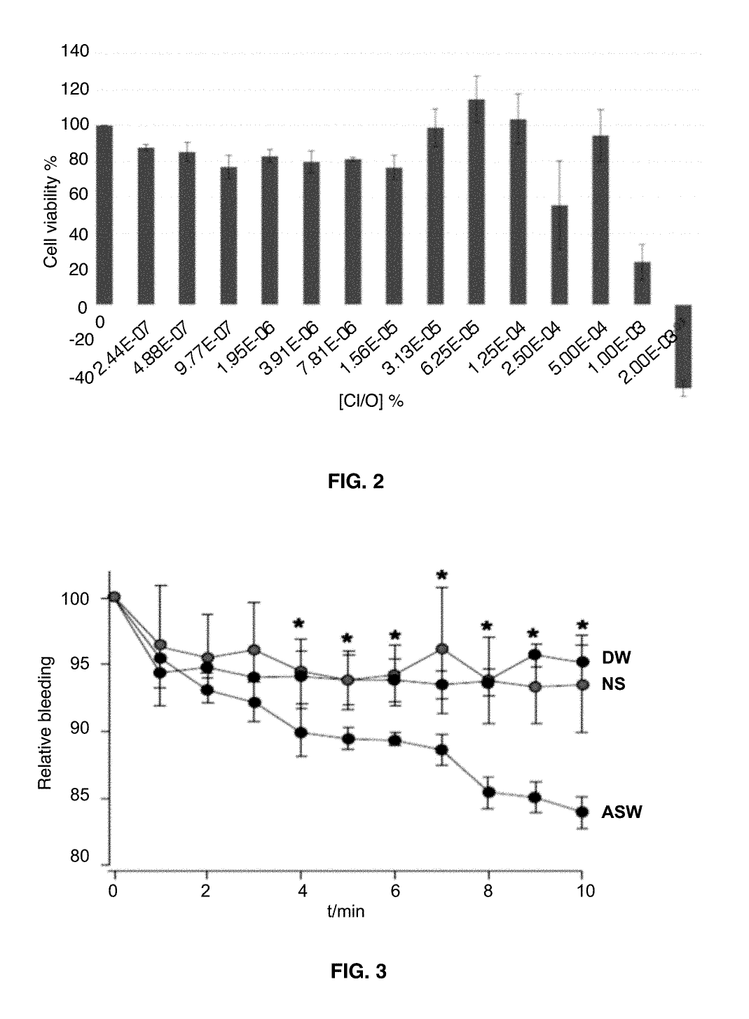

The cytotoxic effect of activated saline water for longer exposure times with lower concentrations of [Cl/O] was evaluated. Confluent cultures of human fibroblasts, obtained from a patient, were used for the test. Cells were grown in culture plates previously packed with coverslips and incubated at 37 degrees C. in 5% CO₂ atmosphere with DMEM culture media. Serial dilutions of activated saline water, starting from a [Cl/O]=20 ppm (0.002%), were made with DMEM and cells were exposed under previously described incubation conditions for 72 hours. Viability was evaluated with Alamar blue. Tests were performed three times. Results clearly show that the highest concentrations of activated saline water reduced viability meanwhile the lowest concentrations keep a comparative cellular viability with control. An outstanding fact is that some concentrations of activated saline water increased cellular density. Thus, activated saline water induces a proliferative effect over cells when administered in proper concentrations of [Cl/O], which are rather low (<1.5 ppm; FIG. 2).

### Example 3

### In Vivo Genotoxicity, Cytotoxicity and Acute or Sub-Acute Oral Toxicity of Activated Saline Water in Wistar Rats and In Vitro Genotoxicity and Mutagenicity of Activated Saline Water

In vitro evaluation of different concentrations of activated saline water genotoxicity was performed according to AMES methodology (OCDE 471) with negative results under experiment conditions. The oral toxicity of ASW was evaluated according to 407 OCDE protocol, using young (4 weeks old) male and female Wistar rats that were monitored until the mature stage to register adverse effects in behavior or growth and in diverse biomarkers of oxidant stress and toxicity. In an acute regime the oral (plastic probe) administration of activated saline water (2 mL/100 g body weight, which is the maximum volume of an aqueous solution that can be administered to the rodents) was 1 time per day during three days. In a sub-acute regime, administration was 1 time per day during 30 days. Animals were kept under controlled temperature (21 deg), humidity (60%) and light-darkness cycles of 12 hours. Seven groups of 10 rats (5 males and 5 females) were treated with: 1) saline (control group), 2) carbon tetrachloride (positive control to oxidant stress), 3) benzene (positive control of genotoxic damage), 4) activated saline water with [Cl/O]=40 ppm, 5) activated saline water with [Cl/O]=20 ppm, 6) activated saline water with [Cl/O]=10 ppm and 7) activated saline water [Cl/O]=2 ppm. These concentrations of [Cl/O] correspond to an administration of 80, 40, 20 and 4 mg of active species of chlorine and oxygen, respectively.

Overall, no apparent alterations in behavior, weight gain or height were reported. No animal died during administration of chemicals or from symptomatology following up. Activities of phase I and phase II enzymes, involved in xenobiotic metabolism like CHCl₃ formation, were reported as normal, suggesting that no chloroform (trihalomethane) was formed under experimental conditions. Serological biomarkers showed that all activated saline water concentrations induced oxidant stress to some extent and that females were more sensitive than males during acute exposition. For females, significant reduction of GSH/GSSG rate was observed only with the two lowest concentrations. In males only the administration of 40 mg induced a significant effect. It is important to note that the proportion of GSSG versus GSH never was higher than 1.5%, while for the group treated with CHCl₃ this value reached 10%. TBARS formation was not significant during the acute regime. No significant changes were observed in the activity of anti-oxidant enzymes catalase, glutathione reductase and glutathione peroxidase but significant reductions in SOD activity was registered in male groups treated with 40 or 10 mg or females treated with 20 and 40 mg.

Sub-acute regime induced significant reduction of GSH/GSSG rate in both genders only with the highest concentrations, but this reduction did not exceed a maximum value of 3.5%. Males were more sensitive than females about lipid peroxidation. TBARS formation was significant in male groups treated with the three lowest concentrations. For this regime, only the lowest dosage induced significant reduction of SOD activity in females. Catalase activity was significantly reduced for both genders with a dosage of 20 mg.

Evaluation of genotoxicity and cytotoxicity in bone marrow showed that activated saline water is weakly genotoxic and cytotoxic for males, but such toxicity is not dosage dependent. No data of these types of toxicity were found in animals treated with activated saline water during the sub-acute regime. Histopathological studies of hepatocytes from males and females exposed to activated saline water during the acute regime revealed slight steatosis for all treatments, without a dose-dependant effect. Surprisingly, the same analysis in animals from the sub-acute regime showed no steatosis data and, moreover, healthy tissue was observed comparatively with the group treated with benzene. Previous results suggest that the organism may adapt to mild oxidant stress stimuli induced by activated saline water components without experiencing cumulative damage. Such stimuli could induce beneficial effects, for example, regeneration of tissues, just as was shown with rodent hepatocytes.

### Example 4

### Analgesic, Anti-Inflammatory, Hemostatic and Regenerative Effect of Activated Saline Water in Murine Models

To evaluate the hemostatic effect of activated saline water, a surgical lesion in the premaxilla region was induced in 45 young Wistar rats of a similar weight and size, under the influence of phenobarbital. Animals were divided in 5 groups of 15 members each. The immediate bleeding was collected over 5 minutes and after that lesions per group were irrigated with: a) normal saline (control), b) activated saline water with 20 ppm of [Cl/O] and c) distilled water. After irrigation, bleeding was determined by collecting the blood with a pre-weighed filter paper each minute during ten minutes. Each paper was weighed, data were collected and statistical analyses of variance and covariance were applied. Results show that activated saline water significantly reduced the bleeding since minute 4, comparatively with normal saline and distilled water (FIG. 3), demonstrating the hemostatic effect of activated saline water.

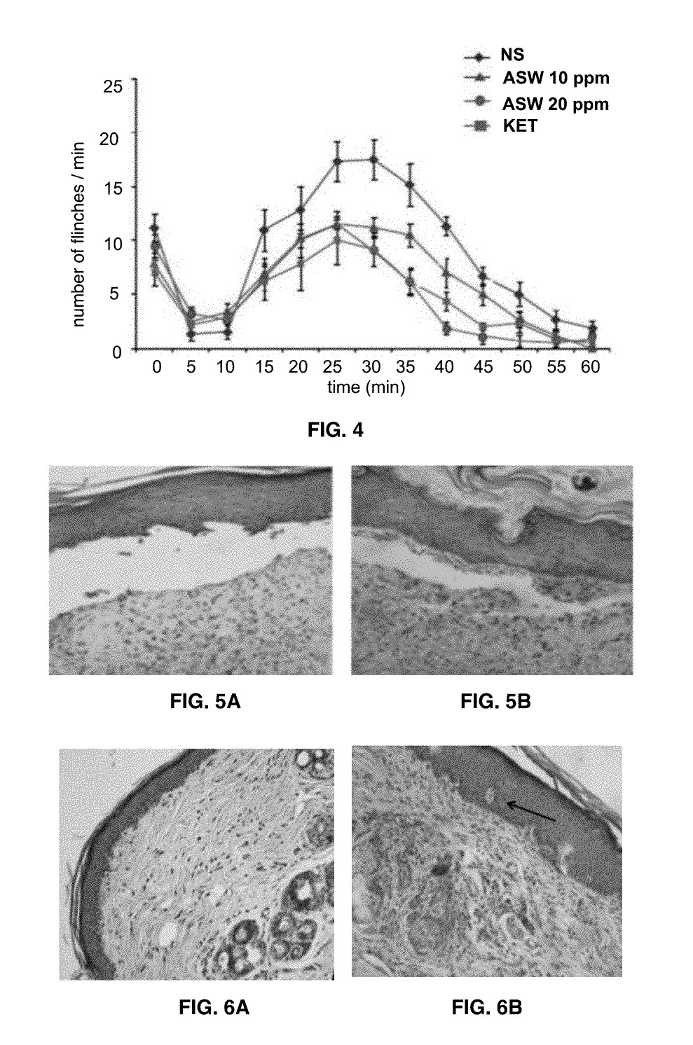

To evaluate the analgesic effect of activated saline water, 40 male Wistar rats weighing 180-200 g were divided into 18 groups of 10 members. Animals were kept at 25 degrees with relative humidity of 60% and under light/darkness cycles of 12 hours. The food was withdrawn 12 hours prior to all experiments, but water consumption was kept ab libitum. The formalin model was used to evaluate activated saline water influence on inflammatory pain. Each animal received 50 mL of 2% formalin solution in the left back paw and the nociceptive response was registered during 60 minutes. 5 minutes previous to treatment with formalin, animals from each group were treated subcutaneously with 50 mL/paw of a) normal saline (positive control), b) activated saline water with [Cl/O]=10 ppm, c) ASW with [Cl/O]=20 ppm and d) ketorolac (100 mg/paw). Results show that ASW with [Cl/O]=20 ppm reduced inflammatory pain in almost 50% of animals, which is the observed value for NSAID Ketorolac. Activated saline water with [Cl/O]=10 ppm also showed an effect close to 35% of pain reduction (FIG. 4). Statistics were performed by ANOVA and Tukey (p<0.05) analysis.

Activated saline water also showed great potential to induce fast and efficient tissue regeneration in vivo, especially when administered as a gel. Under the influence of Zoletil (0.2-0.3 mL/Kg I.V.), 30 New Zealand rabbits were grouped in 6 groups of 5 members and surgical intervention was performed to generate an oval tissue flap (approx. 1 square centimeter) from an internal part of the auricle. The flap was completely devascularized and irrigated per group with a) Normal saline (NS) or b) activated saline water gel with [Cl/O]<10 ppm, as well as irrigating the injury niche. Then the flap was grafted again on the original area of the auricle. The injury was immobilized and covered with sterile gauze to perform a histological following of its evolution. Three groups of rabbits received the same treatment so biopsies (taken from the graft) could be analyzed, per group, at 24 hours, 14 days and 19 days. Animals were irrigated, with a corresponding treatment, 2 times a day for 7 days. Biopsies at 24 hours just revealed inflammatory infiltrate, which is part of natural evolution of lesions, in both groups. Nevertheless the macroscopic aspect of injuries was significantly less red and swollen in animals treated with activated saline water gel. Biopsies of 14 days showed the differences in injury evolution. Animals treated with normal saline presented inflammatory infiltrate, dense collagen fibers and tissue discontinuities (FIGS. 5A-5B) while animals treated with activated saline water gel showed less inflammatory infiltrate, angiogenesis process and regeneration of hair follicle (FIGS. 6A-6B). Treatment with activated saline water gel promoted tissue regeneration more efficiently than normal saline.

### Example 5

### Clinical Evidence of Anti-Inflammatory and Analgesic Effect of Activated Saline Water

Four patients with bone exposure and necrosis associated with bisphosphonate use were treated with activated saline water, as mouthwashes and tooth gel, to alleviate pain, decrease inflammation and control chronic infection. Previous treatments with broad-spectrum antibiotics and NSAIDs had become ineffective in eradicating infection and pain, so alternative treatment was implemented. All patients were treated with activated saline water and activated saline water gel three times a dairy. All patients had an improvement of 90% in anti-inflammatory and analgesic effect comparatively with the use of NSAID alone. Also the infection was eradicated or controlled, contributing to alleviation of pain and inflammation. In conclusion, activated saline water induces anti-inflammatory and analgesic effect in patients.

### Example 6

### Clinical Evidence of Hemostatic, Anti-Inflammatory and Increased Tissue Regeneration Effects of Activated Saline Water

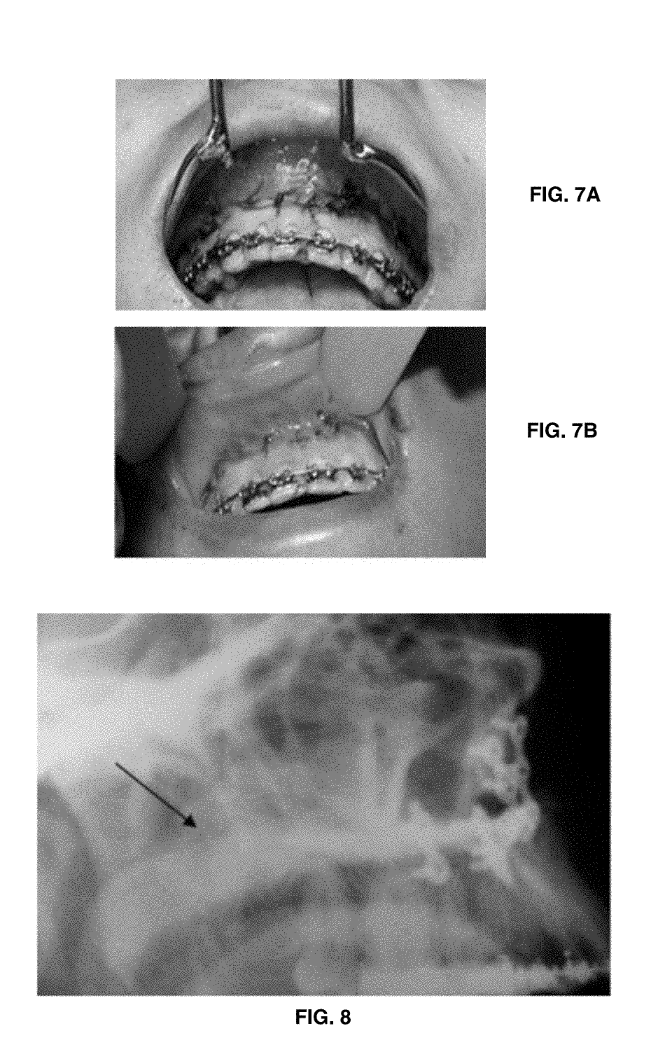

Twenty-five male or female patients with ages between 18-55 years old experienced maxillofacial surgery procedures, i.e., ortognatic surgery or facial trauma managements of similar severity. Surgery on ten patients was conventionally conducted using normal saline (NS) as irrigant, while in fifteen procedures normal saline was substituted with activated saline water ([Cl/O]=20 ppm). In ortognatic surgeries (electrosurgery, electric knife coagulation), patients managed with activated saline water bled 15-20% less than those irrigated with NS. Facial trauma managements with activated saline water showed 50-60% less bleeding. Surgical blood loss was calculated by evaluating differences in amounts of fluid used to irrigate vs volume of fluid extracted with a vacuum pump. For both types of maxillofacial surgeries, patients treated with activated saline water presented with 50-70% less post-surgery inflammation than those managed with normal saline. This effect resulted in a more efficient recovery, for example, by reducing times for tissue regeneration or wound healing. FIGS. 7A-7B and 8A-8D show the efficient recovery of a female patient, 30 years old, that experienced Lefort 1 surgery for maxillary advancement and was irrigated with activated saline water instead of NS.

### Example 7

### Clinical Evidence of Antiseptic, Anti-Inflammatory and Increased Tissue Regeneration Effects of Activated Saline Water

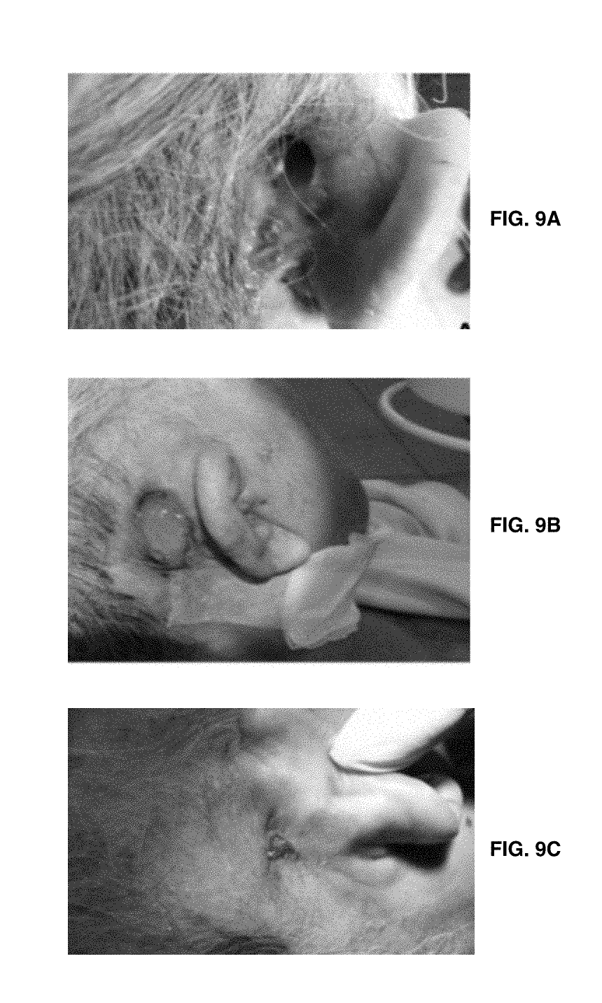

Case 1: A female patient, 71 years old, was treated for acute retroauricular infection (FIG. 9A). Without treatment with antibiotics, the wound was irrigated with activated saline water [Cl/O]=20 ppm and filled with activated saline water gel (FIG. 9B), every three days for the first 2 weeks and two times a week during the remaining time. The wound healed twenty days post-initial treatment with activated saline water (FIG. 9B).

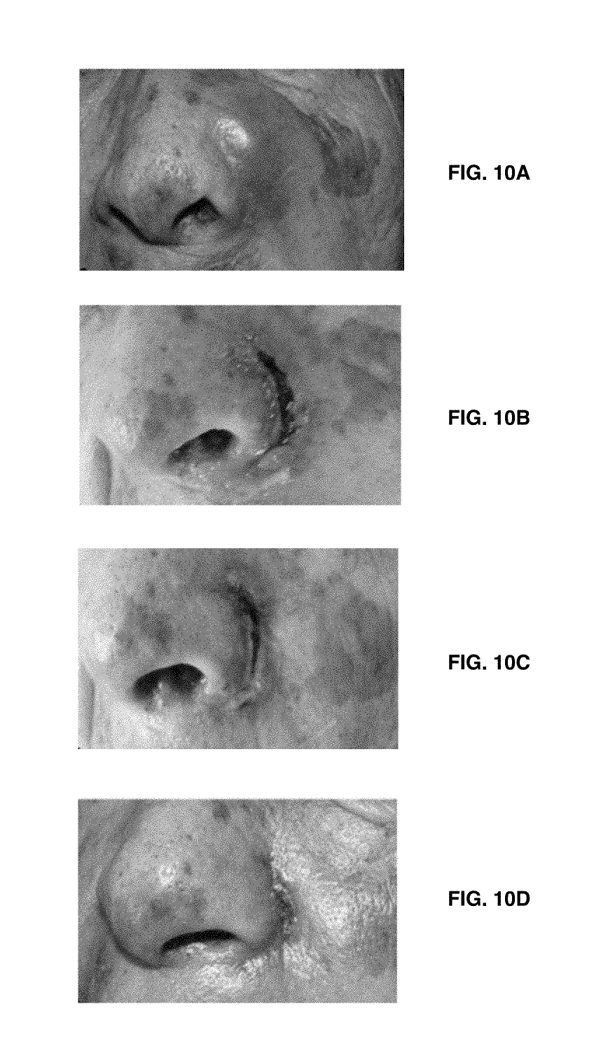

Case 2: A female patient, 84 years old, was treated for an infected apical cyst in the left nasolabial region (FIG. 10A). Diabetic and multi-allergies to antibiotics, she was treated only with activated saline water lavages of the wound (every three days) and was treated with activated saline water after cyst removal. Suture dehiscence is presented 24 hours post-surgical process, so the activated saline water gel was used to fill in the cavity (FIG. 10B). Treatment was performed every 3 days and showed remarkable advance within 9 days (FIG. 10C). The wound healed 21 days after cyst removal (FIG. 10D).

### Example 8

### Clinical Evidence of Intra-Neural Administration of Activated Saline Water to Treat Trigeminal Neuralgia and Bell Palsy

Case 1: A female patient, 67 years old, has a trigeminal neuralgia diagnosis of 5 years of evolution. She is multi-treated with carbamazepine and gabapentin medication without effective pain alleviation. She experienced paroxysmal acute and severe pain as well as electric shock sensation in V2 and V3 zone of right upper lip (trigger zone). Upon signed consent she accepted alternative treatment with activated saline water. There was previous asepsis of the areas and 1.8 mL of activated saline water were administered intra-neurally (V2 and V3 regions of mandibular and maxillary nerves) weekly for 5 weeks. Since the second administration she decided to quit gabapentin and just ingested half of her carbamazepine dose. After the fifth administration she stayed asymptomatic and without oral medication.

Case 2: A female patient, 39 years old, with a typical trigeminal neuralgia diagnosis of 4 years of evolution. She was administered carbamazepine (200 mg each 24 hours) for 2 years to effectively treat pain, but is currently in pain due to the lack of effectiveness of carbamazepine, ketorolac, gabapentin, tramadol or lyrica. She consented to alternative therapy with activated saline water. There was previous asepsis of the areas and 1.8 mL of activated saline water were administered intra-neurally in V3 and V2 (trigger regions) weekly for a month. She gradually quit analgesics and currently remains asymptomatic.

Case 3: A male patient, 53 years old, with trigeminal neuralgia with 25 years of evolution. He was multi-treated with carbamazepine, gabapentin and multi-vitamins, and presenting less and less response to drugs. He consented to alternative treatment with activated saline water intra-neurally in the right infraorbital nerve, the trigger zone in the right side of the upper lip, the low-right premolar region and the mandibular nerve. There was previous asepsis and under simple mepivacaine hydrochloride, 1.8 mL of activated saline water were applied in each zone each of two days during the first week and then weekly for 5 more weeks. The patient reported ceasing treatment with analgesics in week three of the alternative treatment. He was asymptomatic for 2 months and then additional administrations of activated saline water were followed as previously described.

Case 4: A female patient, 34 years old, with no allergies. She presented with left side Bell palsy and asked for the alternative treatment with activated saline water. There was previous sepsis and 1.8 mL of activated saline water were administrated into the anterior lobe of the auricle facial nerve (temporofacial ramus, mandibular ramus and zygomatic ramus regions). Instantly, the patient reported sensitivity and sensation in treated areas, as well as muscular relaxation and release of tears. She was followed monthly for a year without presenting any symptoms.

### Example 9

Alleviation of Severe Arthralgia and in Patients with Chronic Chikunqunya or Rheumatoid Arthritis by Intravenous Treatment with an Activated Saline Water

A clinical study was conducted including 20 patients with chronic Chikungunya (15 patients) or rheumatoid arthritis (5 patients). All patients agreed to participate under informed consent. All patients were clinically and serologically (blood sample) evaluated previously and during the entire study (30 days).

Patients with positive diagnosis of RA or Ch-Chik (with PCR confirmed acute infection and extended symptoms for more than 3 months) and experiencing severe arthralgia, were included in the study. Four groups with 5 members were formed: 3 groups with Ch-Chik patients and 1 group with RA patients. They were all electrocardiographically and serologically (biochemical profile) tested and also clinically evaluated for asthenia, fatigue and pain using standardized questionnaires with international scales like RAPID3. The study lasted 30 days and the tests were applied on days 0, 1, 2, 3, 4, 6, 14 and 28. The activated saline water was administered to all groups, each 24 hours the first three days of study. Different dosages were selected for each group of Ch-Chik: 5 mL to group 1, 10 mL to group 2 and 20 mL to group 3. RA group was treated with 20 mL.

General results showed that intravenous administration of activated saline water induced eradication or significant reduction of mild and severe Ch-Chik or RA symptomatology, especially arthralgia and joint inflammation in patients from all groups, 24-48 hours after administration. It was also shown that activated saline water is safe, since it only induced mild self-limiting adverse effects like headache and somnolence. Biochemical markers diminished by activated saline water administration were fibrinogen and ESR. The best dosage for Ch-Chik treatment was 10 mL of activated saline water each 24 hours for 2 days and, if necessary, additional 20 mL each 24 hours for two more days. On the other hand, treatment of RA depends on its severity so 20 mL of activated saline water each 24 hours for five days were necessary to alleviate symptoms for 1 month. After that time, another cycle of treatment is recommended.

### Example 10

### Clinical Evidence of Immune System Modulation and Hepatic Tissue Regeneration by Intravenous Activated Saline Water Administration

Case 1: A Male patient, 58 years old has a diagnosis of chronic hepatic cirrhosis with esophageal varices. Doppler ultrasound of liver and biliary ducts reported hepatic nodularity with decreased right lobe and hypertrophy of caudate lobe. Heterogeneous echogenicity due to diffuse areas and formation of a pseudo-mass that displaces and separates supra-hepatic veins was found. No data of solid focal lesions. Hepatic hilum with multiple periportal vessels, monophasic hepatopetal flow and venous pattern is present. Hepatic artery had a biphasic flow and picosystolic velocity of 33 cm/sec. The caliber of the suprahepatic veins is diminished and right and left veins are displaced towards the periphery and with three-phase flow. A re-canalized and thin paraumbilical vein is identified in real time. Non-dilated intra- and extrahepatic biliary tract, the vesicle with thickened wall at the expense of thick vessels with venous flow to the Doppler study. No calculi were detected nor pain during palpation of vesicular area. Enlarged spleen with diameter>16 cm, regular borders and homogenous echogenicity. Data of normal kidneys and pancreas. Cavernomatous portal transformation, associated with perivascular varices and splenomegaly, which is congruent with chronic liver cirrhosis.

The patient agreed to alternative treatment with activated saline water, 20 mL intravenous, every 24 hours for 5 days and then weekly for 5 months. The patient reported a life quality improvement including normalization of liver profile enzymes and blood biometry. Esophageal varices disappeared 4 months post-initial treatment with activated saline water. The patient also reported better sleep quality, energy and mood. The new liver and biliary ducts Doppler ultrasound showed: Normal liver, with discretely nodular contours, heterogeneous and discretely echogenic, parenchymatous echogenicity with no evidence of lesions occupying solid or cystic space, with adequate definition of portal and suprahepatic vessels, which with color Doppler application show adequate flow saturation. In the portal vein, multiple collateral images are identified, suggestive of secondary changes to cavernomatous degeneration of the portal, with adequate flow saturation of collateral vessels. Without evidence of dilatation of intra- and transhepatic bile ducts, common bile duct with a diameter of 4.5 mm in its light. Hepatic artery with biphasic spectrum and systolic peak of 64 cm/sec. Suprahepatic vessels with biphasic spectrum and mean flow velocity of 20 cm/sec. Collateral portals with spectra in bar, without modifications to the respiratory cycle and average flow velocity between 36-41 cm/sec. Spleen with normal form, regular counterparts, dimensions 171×132×82 mm in its three axes and echogenicity of the homogenous splenic parenchyma. Portion of splenic hilum with adequate identification of permeable splenic vein with 13 mm caliber, arterialized spectra and average flow velocity 20 cm/sec and some perisplenic collaterals. Normal data of kidney and pancreas. The patient reported the same benefits after 2 years of treatment.

Case 2: A female patient, 45 year old, has a chronic C hepatitis diagnosis, derived from multiple blood transfusion. When she was 5 years old, she was run-over by an automobile, resulting in a hip fracture, involvement of kidney and viscera and nephrectomy. During her recovery, she had recurrent ascites and peritonitis due to drainage, hernias, esophageal varices and internal hemorrhage due to a portal vein fistula, leading to cerebral infarction. A splenectomy was performed with a Warren procedure, Sengstaken-Blakemore catheter placement and varicose vein clearance. As a result, the patient manifested congestive colopathy with varices in the colon and rectum. She received multiple treatments, highlighting continuous steroids. Twenty-eight years after her accident she is diagnosed with hepatic cirrhosis, derived from chronic hepatitis. She currently has 2 pancreatic tumors, a persistent and positive viral count of chronic C Hepatitis, severe costal pain, recurrent evening fever, esophageal varices in the colon and rectum, limited urination (treated with diuretics), depression, lack of concentration and memory, asthenia, and sleep disorders.

She agreed to alternative treatment with activated saline water, 20 mL, i.v. for 7 days, and then once a week for 7 months. The patient reported significant improvement, with no data of fever or costal pain, being able to achieve long periods of deep sleep and adequate urination without diuretic intake. The status of her hepatitis has changed to F2 and she displays mental clarity, energy, improvement in memory, concentration and ability to deal with depression and stress. No pancreatic tumors were detected in ulterior ultrasound study.

### Example 11

Clinical Evidence of Immune System Modulation and Renal Restoration from a Diabetic Patient with Renal Insufficiency

A male patient, 51 years old, type II diabetic with 15 years of evolution and anemic, hypertensive, immunocompromised and with renal insufficiency complication with two years of evolution. He received treatment with glibenclamide+metformin since 15 years ago and with linagliptin since the renal insufficiency diagnosis. A month ago he also started haemodialysis (3.5 hrs) 3 times a week. He experienced exhaustion, fatigue and depression. He presented with low values of erythrocytes, leukocytes, hemoglobin, hematocrit, and glomerular filtration rate (14.66 mL/min/1.73 m²). He also presented with increased levels of cystatin C (3.73 mg/L), glucose (120 mg/dL), creatinine (15.3 mg/dL), ureic nitrogen (110.9 mg/dL), uric acid (8.5 mg/dL), total cholesterol (204 mg/dL), triglycerides (249 mg/dL) and alkaline phosphatase (134 Ul/L).

He accepted alternative treatment with activated saline water. 20 mL of activated saline water was administered intravenously each 24 hours for 7 days and then weekly for 12 months. After the first week of treatment, he was still anemic, but his immune system reached stability with a normal level of leucocytes. Cholesterol (249 mg/dL) and alkaline phosphatase (141 Ul/L) slightly increased but triglycerides (238 mg/dL), glucose (110 mg/dL), uric acid (5.7 mg/dL), ureic nitrogen (43.5 mg/dL) and creatinine (11.3 mg/dL) decreased significantly. He referred to sleeping very well, being in a very good mood and to having energy to walk 30 minutes daily. Three weeks later he changed from three to two haemodialysis sessions per week. Three months after initial treatment creatinine values (10.15 mg/dL) are a little lower and 17 months after the values are 9.3 mg/dL. Glucose, triglycerides, and blood biometry are in normal ranges or very close to reach it. The patient stated that he felt well and that his health has not deteriorated.

### Example 12

### Clinical Evidence of Immune System Modulation, Chemotherapy Detoxification and Bone Marrow Regeneration by Intravenous Activated Saline Water Administration

A male patient, 19 years old, with a right embryonic para-testicular rhabdomyosarcoma (stage IA) diagnosis. His treatment was radical, a right orchiectomy and chemotherapy (actinomycin-D 1 cycle each 21 days, 9 sessions and vincristine, 1 cycle each 8 days). He presented with a dactinomycin (2.5 mg/dosage) overdose, since it was administrated daily during 5 days. Additionally he received 2 doses of vincristine (2 mg each 7 days). Immediately after the last administration he presented with profuse vomiting, nausea, fever (39° C.), diarrhea and right eye diplopy. He was emergency hospitalized and the following was found: Very dry oral mucosa with abundant ulcers on tongue and lateral walls of mouth, as well as saburral secretion, whitish plaques and blistered lesions on the lips. Pale skin and integuments with pustular rashes were on the face (nose, eyelids, malar region), presence of mycotic lesions characterized by whitish plaques, and moldy hair on the nose, forehead and ears. Cylindrical neck without jugular injurgitation. Good tone and intensity of rhythmic cardiac noises and well ventilated lung fields with the presence of scattered bilateral crackles. Soft, depressible, tympanic and painful to palpation abdomen. Suprapubic and genital region with bullous lesions and whitish plaques of fungal characteristics. Permeable vesicle probe with very scarce and concentrated urine output. Hypotrophic limbs. Blood biomarkers data congruent with immunosuppression, thrombocytopenia (values<35×10̂3/m L), leukopenia (values<1×10̂3/mL) and alteration in osmolar and electrolyte values. Initial diagnosis included: Neutropenic fever, Dactinomycin Intoxication Syndrome, Multiple-Spotted Pneumonia of Immunosuppressed Patient, Probably Infectious Enteritis, Oral and Cutaneous Candidiasis, Paralysis of right third-pair, Hydroelectrolytic Imbalance (Severe Hyponatremia, Hypocalcemia and Hypochloraemia) and Right Paratesticular Embryonal Rhytidiosarcoma (AI-I).

Integral treatment was as follows:

A) Protective isolation: Patient was kept inside a highly controlled room that was disinfected three times a day with activated saline water [Cl/O]=40 ppm. Visits were restricted to medical personnel that must satisfy the highest antiseptic standards.

B) Diet: Oral (as possible) and parenteral feeding during 7 days.

C) Treatment of skin and mucous membrane injuries: Lesions were cleaned up with ASW [Cl/O]=20 ppm and covered with activated saline water gel, two times a day.

D) Medication: Omeprazole, 400 mg I.V. each 24 hours; Meropenem, 1 g I.V. each 8 hours for 11 days; levofloxacin, 750 mg I.V. each 24 hours for 11 days; fluconazole, 100 mg I.V. each 12 hours for 11 days; B complex, 1 tab V.O. each 12 hours; filgrastim, 0.3 mcgs SC each 24 hours for 5 days; calcium gluconate, 1 g I.V. each 8 hours; albumin, 1 flask, I.V. each 8 hours; lysine clonixinate, 10 mg, I.V. each 8 hours and dexamethasone, 8 mg, I.V. each 8 hours for 4 days.

During the first 3 days, the patient did not completely respond to conventional medication since blood biometry values did not increase, but reached their lowest values. So family and patient agreed to add that very day, the alternative treatment with activated saline water as follows: activated saline water, 10 mL I.V. each 24 hours, for 10 days.

Evolution of the patient since initial treatment was as follows:

Day 2: Clinical evidence of capillary bleeding. Transfusion of platelet apheresis

Day 3: Resolution of right eye paralysis and the patient referred more energy and less fatigue.

Day 4: Diplopy no longer present.

Day 6: Significant increase and maintenance in with blood cells.

Day 7: Absence of mycotic lesions in skin and mucous membranes and cessation of parenteral feeding.

Day 9: Skin regeneration in face, abdomen, thorax and genitals.

Day 12: Discharged home with controlled following up. His blood biometry values are very close to normality.

Day 17: Blood biometry with normal values. The patient referred no longer symptomatology associated with intoxication.

2.5 years later: The patient presents with no sequels. His spermatic count is normal. There are no precedents in the literature that demonstrate a patient can survive this kind of chemotherapy overdose.

### Example 13

### Clinical Evidence of Masculine and Feminine Idiopathic Infertility Resolution

A couple with a diagnosis of azoospermia and ovarian cysts agreed to alternative treatment with activated saline water. She was 28 years old and type II diabetic in treatment. He was 32 years old without any diagnosis so after 4 days of abstinence, a semenogram was performed. Results are as follows: 5.5 mL of whitish discharge, pH of 8.4, normal viscosity and full liquefaction in 30 minutes. Leucocytes, 8×10 per field; erythrocytes 6-8 per field; zero in the rest of values, implying diagnosis of azoospermia.

Activated saline water administration was performed on both individuals, I.V., 15 mL each 24 hours, six times a week during 15 days and the couple was monitored for 2 more weeks. Blood biomarkers (blood chemistry, blood biometry and liver panel) were normal during the entire treatment. A semenogram immediately after treatment remain the same, but in week 4, germinal cells were detected in the sample. The ultrasound study of the spouse revealed an absence of ovarian cysts immediately after finishing treatment with activated saline water.

### Example 14

### Clinical Evidence of Improvement in Psychological Detriment Due to Exacerbated Stress and Fatigue

Case 1: A female patient, 36 years old, is sedentary, type 1 obese and has metabolic syndrome. She refers to chronic fatigue, discouragement or depression and recurrent insomnia starting a year ago. She agreed to treatment with I.V. activated saline water, 20 mL, weekly for 2 months. Since the first week she reported having effective periods of sleep, an improvement in general mood, higher mental clarity and energy to exercise regularly.

Case 2: A 64-year-old male patient. Controlled asthmatic with eventual episodes of hypertension. He referred to having recurrent headaches, chronic fatigue, apathy associated with exacerbated episodes of stress, and an impossibility to sleep well. He agreed to treatment with I.V. activated saline water, 20 mL weekly for 2 months. The patient showed greater mental lucidity, better sleep and mood, energy to perform his daily tasks, as well as a more relaxed attitude in acute stress situations and headache remission. Additionally, the patient reported no hypertensive episodes during treatment and better a response against colds.

### Example 15

Clinical Evidence of Improvement in Energy Levels after Intense Training in High Performance Athletes

Five kick boxing high performance athletes agreed to alternative treatment with activated saline water to resolve acute fatigue and lack of energy. They had very high intensity training during 5 days and on the fourth day they manifested acute fatigue, low mood, and low performance during training. 10 mL of I.V. activated saline water was administered at the end of the day. On the next morning they all reported high energy, due to deep sleep, and to mental wellness and could complete their 5 days regimen of intense training.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. It is therefore evident that the particular illustrative embodiments disclosed above may be altered or modified and all such variations are considered within the scope and spirit of the present invention.

What is claimed is:

1. A neutral electrolyzed water that induces modulation of immune system, haemostasis, tissue repair, analgesia and an anti-inflamatory effect, comprising:
in reverse osmosis water:
an active oxidant species of chlorine and oxygen [Cl/O] comprising HClOₓ, wherein X is 1 to 4, ClOₓ⁻, wherein X is 0 to 4, ClO₂, O_{y}, wherein Y is 2 or 3, Cl₂, H₂, or H₂O₂, or combinations thereof at a concentration of about 0.00001% or 0.1 ppm to about 0.004% or 40 ppm; and
sodium chloride at a concentration of about 3% or less.

2. The neutral electrolyzed water of claim 1, wherein a pH thereof is about 6.5 to about 7.5.

3. The neutral electrolyzed water of claim 1, wherein an oxidation-reduction potential thereof is about 500 mV to about 980 mV.

4. The neutral electrolyzed water of claim 1, wherein an osmolarity thereof is about 30 mOsm/L to about 350 mOsm/L.

5. The neutral electrolyzed water of claim 1, wherein the [Cl/O] is about 0.001% to about 0.004%, the oxidation-reduction potential is about +750 to about +980 mV and the osmolarity is about 30 mOsm/L to about 100 mOsm/L.

6. The neutral electrolyzed water of claim 1, wherein the [Cl/O] is about 0.0005% to about 0.002%, the oxidation-reduction potential is about +600 to about +900 mV and the osmolarity is about 30 mOsm/L to about 150 mOsm/L.

7. The neutral electrolyzed water of claim 1, wherein the [Cl/O] is about 0.00001% to about 0.001%, the oxidation-reduction potential is about +500 to about +650 mV and the osmolarity is about 150 mOsm/L to about 350 mOsm/L.

8. A formulation comprising the neutral electrolyzed water of claim 1 in a saline vehicle or in a gel.

9. A method for reducing bleeding in a subject during a procedure performed thereon, comprising the step of:
administering one or more times to the subject an amount of the neutral electrolyzed water of claim 1 effective to induce hemostasis therein, thereby reducing bleeding during the procedure.

10. The method of claim 9, wherein the neutral electrolyzed water has a pH of about 6.5 to about 7.5, a [Cl/O] of about 0.001% to about 0.004%, an oxidation-reduction potential of about +750 to about +980 mV and an osmolarity of about 30 mOsm/L to about 100 mOsm/L.

11. The method of claim 9, wherein the step of administering the neutral electrolyzed water induces repair of an injured tissue, reduces inflammation or reduces pain or a combination thereof.

12. The method of claim 11, wherein the repaired injured tissue is skin, connective tissue, muscle, or periodontium.

13. The method of claim 9, wherein the procedure performed on the subject is a surgical procedure, a dental procedure or a medical procedure to treat a lesion, a wound, a burn, or a site of infection.

14. A method for reducing neuropathic pain or for alleviating palsy in a subject in need thereof, comprising the step of:
administering one or more times to the subject an amount of the neutral electrolyzed water of claim 1 effective to reduce the neuropathic pain or to alleviate the palsy in the subject.

15. The method of claim 14, wherein the neutral electrolyzed water has a pH of about 6.5 to about 7.5, a [Cl/O] of about 0.0005% to about 0.002%, an oxidation-reduction potential of about +600 to about +900 mV and an osmolarity of about 30 mOsm/L to about 150 mOsm/L.

16. The method of claim 14, wherein the step of administering the neutral electrolyzed water induces regeneration of a tissue, reduces inflammation or a combination thereof.

17. The method of claim 16, wherein the regenerated tissue is nerve fibers, myelin or myelin axons.

18. The method of claim 14, wherein the subject has a neuralgia, diabetic neuropathy, Bell's palsy, or a viral disease.

19. A method for modulating an activity of the immune system to induce a therapeutic effect in a subject in need thereof, comprising the step of:
administering one or more times to the subject an amount of the neutral electrolyzed water of claim 1 to the subject such that the activity of the immune system is regulated effectively to modulate inflammatory and oxidant profiles, to improve response against stress-related conditions, to stimulate repair of a tissue or an injured organ, to reduce pain or a combination thereof in the subject.

20. The method of claim 19, wherein the neutral electrolyzed water has a pH of about 6.5 to about 7.5, a [Cl/O] of about 0.00001% to about 0.001%, an oxidation-reduction potential of about +500 to about +650 mV and an osmolarity of about 150 mOsm/L to about 350 mOsm/L.

21. The method of claim 19, wherein the tissue or organ is skin, oral tissues, bone marrow, myelin, sexual gonads, kidney, liver, pancreas, muscle, or a combination thereof.

22. The method of claim 19, wherein the subject has chronic chikungunya, rheumatoid arthritis, arthralgia, cirrhosis, hepatitis, diabetes, migraine, fatigue, stress, metabolic syndrome, polycystic ovary, azoospermia, renal insufficiency, esophageal varices, kidney damage, liver damage, medullary hypoplasia, neutropenic fever, lupus, depression, irritable bowel syndrome, obesity, extreme fatigue from physical activity, sleep disorders, asthma, allergies, infertility or a combination thereof.
